(19) United States
(12) Patent Application Publication
(10) Pub. No.: US 2023/0236218 A1
(43) Pub. Date: Jul. 27, 2023

(54) SYSTEM AND METHODS FOR MOTION TRACKING

(71) Applicant: Movella Holdings B.V., Enschede (NL)

(72) Inventors: Joris VOERMAN, San Jose, CA (US); Jasmin HONEGGER, San Jose, CA (US); Ruben LUCAS, San Jose, CA (US)

(21) Appl. No.: 17/581,747

(22) Filed: Jan. 21, 2022

(51) Int. Cl. G01P 15/18; G01P 15/08; G01D 5/353; G01K 13/20

(52) U.S. Cl. CPC G01P 15/18; G01P 15/08; G01D 5/35316; G01K 13/20

(57) ABSTRACT

A system includes a first and second sensor for a first and second body portion, wherein each sensor includes perturbation sensors for determining physical perturbations for each respective body portion in response to a body movement, each sensor includes a processor for determining orientation and velocity data for each respective body portion in response to the physical perturbations, a shape sensing unit coupled to a joint portion between the first and second body portions including an optical fiber configured to bend in response to the body movement, a light source for providing light into the optical fiber, a light sensor for sensing reflected light from the optical fiber in response to being bent, and a processor for determining curvature data associated with the joint portion, and a central processor for determining user movement data in response to the orientation and velocity data for each body portion and the curvature data.

### BACKGROUND

This disclosure generally pertains to motion tracking. More particularly, various embodiments disclose systems and methods for motion tracking utilizing inertial measurement (IMU) devices and shape-sensing optical fibers.

Human body motion tracking has important applications in many fields, such as medical, biological science, animation, etc. Currently a popular method for tracking human motion tracking is via optical based tracking. These systems typically include a series of optical markers placed on a human body, a series of high-speed cameras that are able to capture images of the optical markers in two-space, and a processing unit that triangulates the positions of these markers into three-space. Disadvantages of such systems include that they require dedicated image capturing studios, they are very labor-intensive to configure and setup, they require high amounts of imaging and processing capability, and the like. Additional disadvantages include, when tracking the body of a performer, the performer has to wear a special marker suit and cannot wear a normal costume, and when tracking a face of the performer, the performer has to tolerate a series of dots being stuck or painted on their face while they perform. Accordingly, such camera and marker-based solutions are expensive and very impractical to use for general purposes.

To address such drawbacks, the inventors of the present invention have been on the forefront of commercializing the use of inertial measurement unit (IMU) based motion tracking systems. Such IMU systems require placement of multiple IMUs upon a performer, e.g. under their costumes, and allow the performer to perform in any environment, e.g. outdoor settings, impromptu settings, etc. The movement data that is captured is then combined with physiological information known about the performer to determine performer movement.

The inventors recognize that there are certain types of movement that are challenging to capture with IMU motion tracking. This includes twisting of portions of the body, slow motion movements, fine-grained motions, and the like. The inventors have contemplated solutions that use larger numbers of IMUs to capture and precisely track bends/twists of the body, such as movement along the spine. However, the inventors believe that drawbacks to these solutions may include that it is hardware intensive, requires very high data bandwidth, and requires very high data processing capability.

In light of the above, there is a need for a solution that can provide precise and accurate translation of the poses of subject (e.g. a human body, a machine, a vehicle, etc.) without the drawbacks described above.

### SUMMARY

The present invention relates to systems for motion capture. More particularly, embodiments of the present invention relate to systems for enhanced motion capture based upon inertial measurement units (IMUs).

Various embodiments described herein include systems that integrate shape sensing technologies and IMU-based systems. Additional embodiments include methods that provide estimates of the fine movements of the human body segments and joints in an ambulatory environment. In some embodiments, an integrated system includes multiple IMU(s) and optical fiber(s) with Fiber Bragg Grating(s) (FBGs). Data obtained therefrom are then integrated together to provide high-quality motion data.

In some embodiments, IMUs (e.g. including gyroscopes, accelerometers, or the like) are typically attached to rigid portions of a performer's body (e.g. arms, legs) and the mounting locations are recorded. These rigid portions of the body (e.g. segments) are typically separated by a joint or other flexible region. In some embodiments, optical fiber(s) may also be attached to rigid portions of the performer's body, wherein portions of the optical fibers with a characteristic geometric structure (e.g. Fiber Bragg gratings (FBGs))extend across the joint or other flexible region. Herein, the optical fibers together with FBGs may be termed an FBG device. An optical sensing unit is coupled to the FBG device and may send light (e.g. a laser beam) to the FBG device, and may receive reflected light from the FBG device. Herein, the combination of the FBG device and optical sensing unit may be termed a shape sensing device.

In operation, as a performer moves, data is simultaneously recorded in the IMUs and the shape sensing device. For example, the IMU sensing units measure the acceleration and angular velocity of the performer's movements, and using Kalman filtering or other available algorithms, the initial estimated positions of the IMUs and segments are determined. At the same time, the shape sensing device outputs a laser through the optical fiber of the FBG device, and as the performer bends, the reflected light returned from the FBGs will have wavelengths that characterize the bending. The physical curvature data of the FBG device is then determined by the reflected wavelengths using Frenet-Serret equations or other available algorithms. Subsequently the initial estimated positions of the segments are processed along with the physical curvature data to determine the refined estimated positions of the segments.

### BRIEF DESCRIPTION OF THE DRAWINGS

In order to more fully understand the present invention, reference is made to the accompanying drawings. Understanding that these drawings are not to be considered limitations in the scope of the invention, the presently described embodiments and the presently understood best mode of the invention are described with additional detail through use of the accompanying drawings in which:

FIG. 1 illustrates an example diagram according to some embodiments;

FIG. 2 illustrates a block diagram according to some embodiments;

FIG. 3 illustrates a block diagram according to some embodiments; and

FIG. 4 illustrates a flow-chart diagram of a processing pipeline according to some embodiments.

### DETAILED DESCRIPTION

Motion tracking typically involves sensing motions of a performer and determining skeletal poses (or shape) of the performer. More specifically, motion sensing attempts to determine how the segments (e.g. portions of the performer, user or subject), or how a collection of segments move in space, whereas shape sensing attempts to determine how the segments or linked segments are oriented relative to each other. Various embodiments described herein describe a unique hybrid motion capture system.

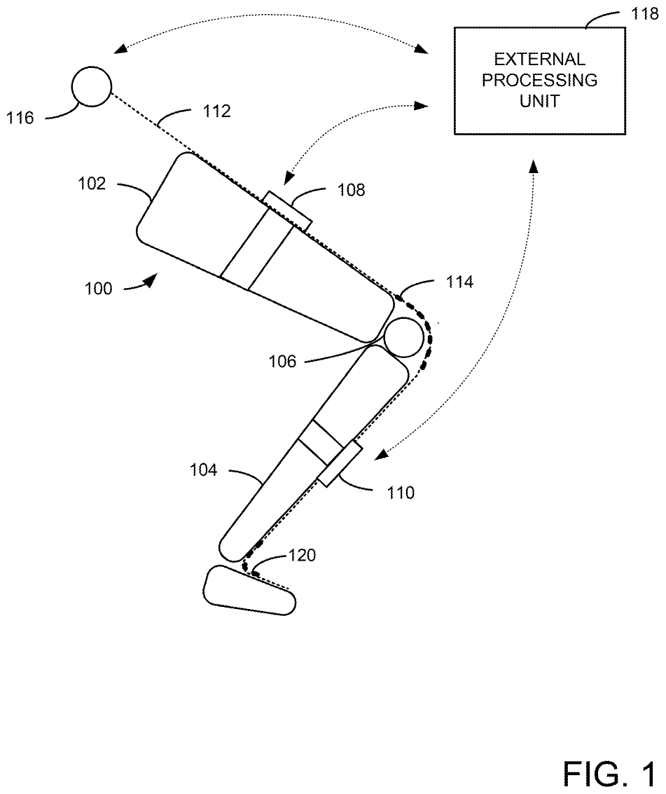

FIG. 1 illustrates an example configuration according to some embodiments. More specifically, FIG. 1 illustrates a performer 100 including segments 102 and 104 that are coupled via a joint 106. Also illustrated is a first inertial measurement unit (IMU) 108 coupled to segment 102, a second IMU 110 coupled to segment 104, and an optical fiber 112 disposed over joint 106. IMUs 108 and 110 may be attached to the performer via elastic straps, Velcro, sockets in a garment, or the like. In some embodiments, optical fiber 112 may be disposed in a sleeve, or the like, which in turn is adhered across the performer's joint 106. Optical fiber 112 may include a portion 114 that is characterized by the geometric patterning, e.g. Fiber Bragg gratings. Additionally, optical fiber 112 may be coupled to a shape sensing device 116, which can be coupled to or attached to segments 102 or 104, or to other portions of the performer, via optical fiber 112.

In various embodiments, performer 100 may be a human, an animal, or any other object where motion capture is desired, e.g. a robot, a vehicle, or other object. Typically segments 102 and 104 may be geometric portions of an object, e.g. lower leg, upper leg, forearm, sternum, or the like, that typically do not appreciably bend or flex. Joint 106 may be any type of flexible coupling member coupled between segments 102 and 104 that allow segments 102 and 104 to bend, twist, compact, or the like relative to each other.

In various embodiments, motion tracking systems provided by Movella, the assignee of the present application are used. These inertial measuring unit (IMU) systems, e.g. 108, typically include three-dimensional accelerometers, three-dimensional gyroscopes, a processor, and a wireless-transmitter that are attached to portions of the performers body, e.g. hip, hand, or the like. In some embodiments, the systems may also include functionality such as magnetometers, pressure sensors, temperature sensors, and the like. Additionally, these systems may include biomechanical software constraints to help ensure that anatomically correct shapes for the performer are respected when determining output data for each segment. For example, if alternative orientations for segments are possible, the orientation that is anatomically reasonable may be selected for output, e.g. knees typically bend primarily in one direction. In some embodiments, the output data for each segment may include data such as: orientation data, velocity data, and the like.

Additionally, in various embodiments, shape sensing technology utilizing optical fibers 112 are also used. In some examples, the optical fibers may be single core or multi-core. Further, in some specific examples, the optical fibers are formed including reflective structures 114 (e.g. a regular structure), such as Fiber Bragg Gratings (FBGs), or the like. As discussed herein, a laser beam is input to the optical fiber (by a light source, e.g. 116) and based on the strain experienced by these reflective structures, light of specific wavelengths are reflected back and sensed (by a light detector, e.g. 116). As portion 114 of the optical fiber bends, the specific wavelengths that are reflected are changed. In various embodiments, the wavelength shift depends on the offset of the optical fiber from the neutral axis of bending. As illustrated in FIG. 1, the optical fiber may include a portion 114 that includes a geometric grating, e.g. a Fiber Bragg grating, or the like. In some embodiments, the optical fiber may span more than one joint and include more than one reflective structures, e.g. geometric gratings, as illustrated by portions 114 and 120. This may be implemented by portion 120 having a different grating spacing relative to portion 114, or the like.

In various embodiments, a processor (e.g. in 116) correlates the change in reflected light wavelength to an amount of bending, e.g. induced curvature, of the optical fiber. Such embodiments require calibration prior to use, including 1) placing the optical fiber on a flat surface for zeroing and then 2) placing the optical fiber on a known curvature on which the device is bent. In some examples, the curvature obtained for an optical fiber is used as input to a processor running processing algorithms, such as the Frenet-Serret equations. Such algorithms may be programmed to output a proposed continuous shape of the optical fiber(s) that represents a shape of the human body segment or collection of segments underneath the fiber(s).

In various embodiments, the optical fiber(s) may be coupled to or embedded within an adhesive to create an offset from the neutral axis of bending. In typical application, this optical fiber(s) is then placed over the relevant joint(s), sometimes as close as possible to the skin or surface of the performer or subject. In some embodiments, to avoid the optical fiber from stretching excessively, the optical fiber may be placed into a sleeve such that the optical fiber can slide and move freely during the relevant performer motion. Additionally, the friction between the optical fiber and sleeve is reduced thereby reducing the noise in the measurements..

The above-described embodiments are just one possible hardware configuration. It is expected that one of ordinary skill in the art will recognize that there are many possible configurations that are within the scope of embodiments of the present invention. For example, where and how the optical fibers are attached to physical segments, linked segments, or joints of the performer can vary depending upon engineering preference. Generally, what is desired are hardware that can easily determine changes in reflected wavelengths of light that correspond to changes in the bending and twisting motion of the joints.

In the embodiments illustrated in FIG. 1, to facilitate the integration of data from IMUs (e.g. 108 and 110) and shape sensing unit 116, an external processing unit 118 is provided. As will be discussed below, certain types of algorithms may be processed on-board within IMUs and interrogators, and other types of computationally intensive algorithms may be processed external to these units, by processing unit 118. In various embodiments, IMUs and interrogator units may be coupled to processing unit 118 via a wired connection.

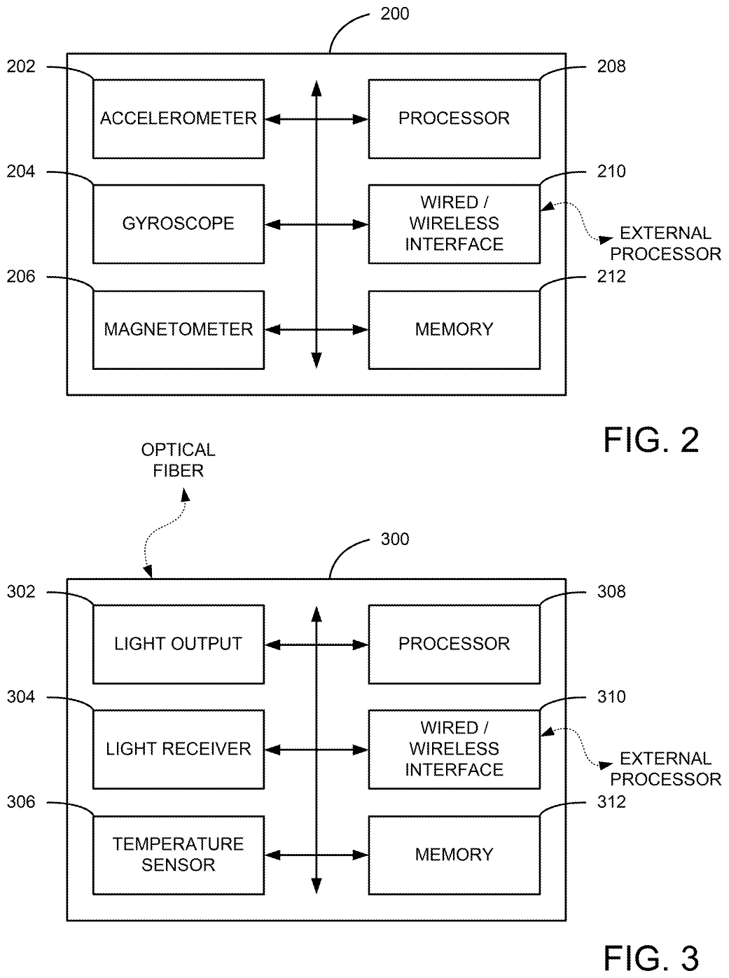

FIG. 2 illustrates a block diagram according to one embodiment. More specifically, FIG. 2 illustrates a block diagram of an inertial measurement unit (IMU) 200, as discussed above. In various embodiments, IMUs may include a 3D gyroscope (202), and a 3D accelerometer (204), which provides angular velocity and acceleration measurements respectively. In some cases, the IMU may also include additional devices such as a 3D magnetometer (206) to provide heading information in the global frame, a pressure sensor, temperature sensors, and the like. In various embodiments, IMUs may also include a processing unit (208), a wired or wireless interface 210, a power supply (e.g. battery), and a memory 212 for storing programs. Such programs may include instances of algorithms that combine high-rate accelerometer data, gyroscope data, and the like based upon the physical movements of the performer, into estimates of the sensor orientation, velocity, or the like.

In various embodiments, the computed data may be transmitted to a remote master processing unit via wired or wireless interface connection, for further processing. By determining the estimates of orientation, velocity, and the like on board the IMU, the amount of data passed from the IMU to the external processing unit is greatly reduced, and thus the data bandwidth requirements is reduced. In various embodiments, the external processing unit typically has higher processing capability, thus computationally intensive algorithms are more appropriately implemented by the external processing unit.

As discussed above, the IMU may include and perform a number of processing algorithms based upon the captured data. In some instances, strapdown integration (SDI)-based algorithms are used, such as those provided by the assignee of the present patent application, to facilitate determination of the orientation and velocity data for a segment. These SDI processes may provide more accurate numerical integrations, especially in cases when the input data are not necessarily synchronous in time.

FIG. 3 illustrates a block diagram according to one embodiment. More specifically, FIG. 3 illustrates a block diagram of a shape sensing device 300, as discussed above. In various embodiments, shape sensing device may include a light (e.g. laser beam) output portion 302, a light sensing portion 304 and a temperature sensor 306. In various embodiments, shape sensing device 300 may also include a processing unit 308, a wired or wireless interface 310, a power supply (e.g. battery), and a memory 312 for storing programs. Such programs may include instances of algorithms that determine an amounts of bending or twisting of an optical fiber based upon the physical movements of the performer, into estimates of curvature data, or the like. In some embodiments, the temperature of the optical fiber or shape sensing device 300 may be used to adjust the estimates of curvature data.

The inventors believe that some types of motion capture of a performer might be determined based solely upon the bending of optical fibers, such as those described above. Such embodiments would require the performer to have optical fibers with optical gratings positioned across most of their joints. Similarly, the inventors believe that some types of motion capture of the performer may be determined solely upon IMUs, by the use of numerous IMUs upon the body. In practice, however, the inventors believe that either solution alone would be limited in the types of motion they can capture. Further, each solution alone would be very expensive solutions in terms of hardware and would require computationally expensive processing solutions. Additionally, it may be difficult for such solutions to provide real-time positional data, that is often required within the motion capture industry. As described herein, to facilitate more complete performer motion capture, embodiments of a hybrid or integrated motion capture system including optical fibers and IMU-based motion tracking are disclosed herein. Such embodiments are believed to reduce the errors and limitations inherent in the different respective motion capture technologies.

In various embodiments, IMUs and shape sensing units may be considered opposites. For example, shape sensing units are based upon optical signals and thus are fully immune to electromagnetic interference, unlike an IMU. Additionally, the rotation and gravitational pull of the Earth do also not affect the curvature measurements and determination capability of the shape sensing units, unlike an IMU. Still further, shape sensing units are typically not prone to errors, such as Nyquist noise, sensor drift, sensor-to-segment calibration errors, soft tissue artifacts, and the like, as IMUs typically are. In contrast, however, IMUs are typically not as sensitive to temperature changes, applied pressures and stretching of the sensors, as shape sensing units are.

In various embodiments, shape sensing units may capture different types of data, that IMUs cannot easily determine, depending on how the optical fibers are attached to the performer. For example, when attached, off-center, to a thin component, the shape sensing unit can measure the bending or curvature of that component. As another example, when attached orthogonally to a source of pressure, the optical fiber may measure a pressure through the Poisson effect. As still another example, through thermal expansion of an optical fiber, the shape sensing units may measure an operating temperature. As discussed above, shape sensing units may be used to measure curvature of a joint of a performer, a machine, a robot, or the like.

In various embodiments, when shape sensing units may be attached to a component (e.g. a joint) and used to measure the curvature of the motion, it is difficult to determine in which direction the fiber was bent, since the resulting signal is simply a wavelength shift. To reduce the ambiguity, optical sensing units may be constrained to joints such the optical fibers will bend primarily in one direction, for example placing it over, next to or close to the knee joint. In practice, joints such as the knee are not perfect hinge joints, since ab-/adduction is present as well flexion/extension motion. Accordingly it is difficult for shape sensing units to accurately measure joint movement. Further, the use of shape sensing units in capturing joint movement is very difficult when attempting to capture bending for more complex joints, such as the ankle or the spine.

In light of the above, the inventors propose a hybrid motion capture system that includes IMUs with biomechanical constraints to reduce the possibility of ambiguity of shape sensing unit data. In practice, the inventors believe this unique combination of shape sensing unit and IMU provides motion capture capability that can rival or is superior to the accuracy and precision of the typical marker-based motion tracking systems mentioned above. Additionally, embodiments are easier to set up, do not require a dedicated motion capture stage, and is more cost-effective.

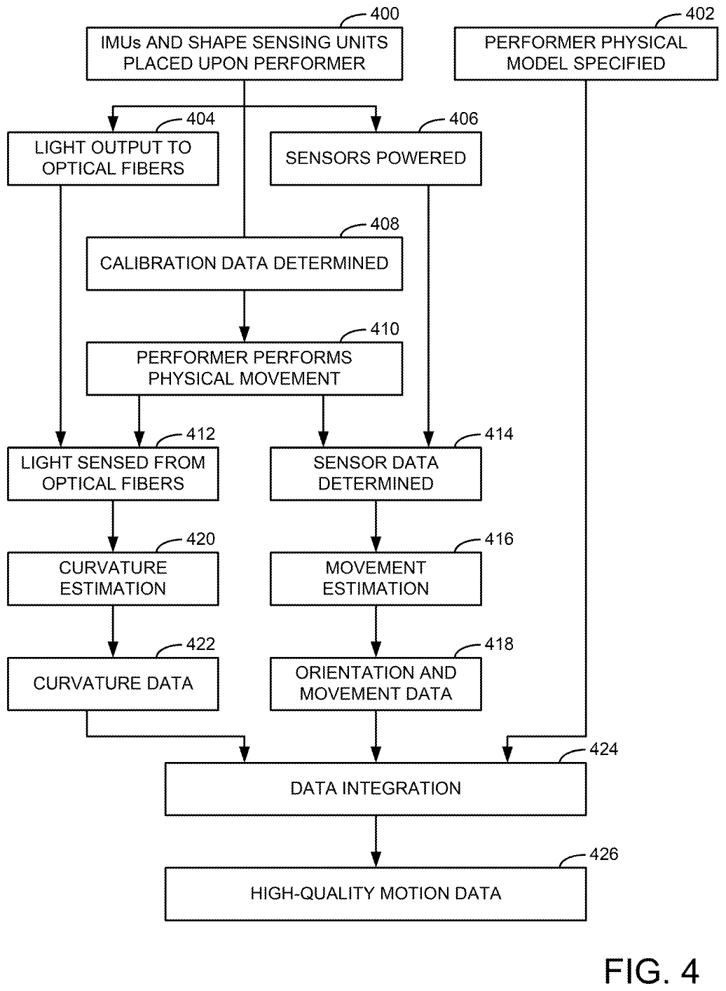

FIG. 4 illustrates a process diagram according to various embodiments of the present invention. Initially, IMUs and shape sensing units are disposed upon the performer, step 400. As discussed above, the placement of optical fibers upon joints, or the like is important to obtain useful data. As part of the set-up process, a physical model of the performer may be specified, including position of particular segments and physical constraints, step 402. For example, models of elbows and knees of a human performer typically are constrained to bend only in one direction, and the like.

In various embodiments, the IMUs and shape sensing units may be powered on. As part of this process, the light output portion of a shape sensing unit may output light to an optical fiber, and the light sensing portion may sense reflected light, step 404. Additionally, the physical sensors of an IMU (e.g. accelerometer, gyroscope, or the like) may be powered on, and begin providing sensed physical perturbation data (e.g. accelerations, rotations, or the like), typically in three-dimensions, step 406. Next, IMUs and shape sensing unit measurements may be made of the performer in a neutral position, and then in specific poses may be captured, step 408. As discussed above, such positions may be used to provide calibration data for the sensors, that will be used below.

In various embodiments, the performer performs physical actions, step 410. For example, a human can jump or dance, an animal may rear-up or run, a machine may operate, and the like. During these performances, the IMUs and sensing units will sense the physical perturbations, for example, by sensing change in reflected light frequency, step 412, and by sensing changes in capacitance, resonant frequency, or the like, step 414.

In response to sensed data, e.g. accelerometer, gyroscope data, magnetic field data, or the like, the sensed data is processed using an estimation algorithm to determine estimates of orientation and acceleration, step 416. In some embodiments, an algorithm such as a Kalman filter, a particle filter or the like may be used. Using the calibration data, the estimates of the orientation and acceleration, the orientation and movement data may be output to the external processor, step 418.

Additionally, in response to the sensed data, e.g. light reflected by geometric structures proximate to a joint, changes in wavelengths are computed into an initial curvature estimation, step 420. Using the calibration data, the estimates of curvature of the optical fiber may be determined and output to the external processor, step 422. In some embodiments, algorithms, such as Frenet-Serret equations can be used to determine the shape of the optical fiber and translates the curvature data into the shape or pose of the linked segment and joint. In an extension of the disclosed principle, shapes of multiple linked segments, such as those on a lower body, are combined to get a more complete pose of the associated body part..

In various embodiments, the external processor receives and processes the shape sensing data and the IMU data. In some embodiments, the sensor data in addition to the physical model of the performer are integrated, step 424. As discussed above, for human performers, the physical model may be a biomechanical model of the human body, i.e. skeleton. In such embodiments, the combination of motion, shape, and mechanical data provide for a more accurate motion capture of the performer, step 426. For example, complex motions of the spine, knees, neck and the like can now be captured.

One specific example implemented by the inventors was for modeling spine movement. In this example, the optical fiber was embedded within a thin component and placed adjacent to and along the spinal column. The grating portion of the optical fiber was firmly affixed at the sacrum within a low friction sleeve, allowing the optical fiber to freely move along the body during motion capture, while staying close to the true curvature of the spine. IMU sensors were placed at the sacrum, and one on each shoulder, approximately on the supraspinous fossa. In this example, the IMUS provided global trunk motion data while the shape sensing device provided measurements of the spinal curvature. Since the shape sensing unit primarily measured the spine in a single dimension, the combination of IMU data and shape data can help to differentiate total spinal motion (i.e., flexion vs. lateral bending).

In various implementations of this integration, each joint in the biomechanical model and constraints on the movement of the two connected segments are formulated. Additionally, the IMU and the shape sensor data provide input on the movement of the segments, by specifying an estimate of an angle of the joint. These inputs, including the biomechanical constraints, are written in the form of a cost function, and a numerical optimization can be used to determine a most likely, e.g. realistic, pose for the performer in each frame. In various embodiments, this process requires many iterations to fine tune the cost functions and framework for each input.

In some embodiments, the high-quality motion data that is determined is then used as input data for computer-generated graphics and models. For example, motion data may be used as a basis for generation of automated characters in a video game, may be used as a basis of characters in an animated feature, may be used for motion studies/ergonomics, and the like.

Further embodiments can be envisioned to one of ordinary skill in the art after reading this disclosure. For example, in some embodiments, multiple optical fibers may be used for a single joint. The data captured by each of these multiple fibers may be used to determine additional movements of a joint, may be used to determine lower noise data, and the like. Additionally, in various embodiments, other types of grating structures may be used for optical fibers than Fiber Bragg gratings, further the periodicity of such gratings may be different. In some embodiments, a first device may be provided coupled to one end of the FBG device for outputting light signals, and a second device may be provided coupled to the other end of the FBG device for receiving the light signals. In such embodiments, the transmitted light (in contrast to the reflected light, above) may be used to facilitate determination of a shape of the FBG device. Further, in some embodiments, the specific algorithms used to determine estimated motions and rotations may be different from those disclosed herein. In some embodiments, the IMUS and shape sensing units may be disposed or sewn into a garment that the performer wears, or may be manually affixed onto the performer. The block diagrams of the architecture and flow charts are grouped for ease of understanding. However, it should be understood that combinations of blocks, additions of new blocks, re-arrangement of blocks, and the like are contemplated in alternative embodiments of the present invention. The specification and drawings are, accordingly, to be regarded in an illustrative rather than a restrictive sense. It will, however, be evident that various modifications and changes may be made thereunto without departing from the broader spirit and scope of the invention as set forth in the claims.

We claim:

1. A system for capturing motion comprising:
a plurality of inertial sensing units comprising a first inertial sensing unit and a second inertial sensing unit, wherein the plurality of inertial sensing units are coupled to a plurality of segments, wherein the plurality of segments comprises a first segment and a second segment, wherein the first inertial sensing unit is coupled to the first segment, wherein the second inertial sensing unit is coupled to the second segment, wherein the first segment is coupled to the second segment via a first joint, wherein each sensing unit from the plurality of inertial sensing units comprises:
an accelerometer configured to output acceleration data in response to a first plurality of physical perturbations;
a gyroscope configured to output gyroscopic data in response to a second plurality of physical perturbations, and
an inertial sensing unit processor coupled to the accelerometer and to the gyroscope, wherein the inertial sensing unit processor is configured to determine orientation data and velocity data for a coupled segment in response to the acceleration data and to the gyroscopic data;
a shape sensing unit coupled to the first segment, the first joint, and to the second segment, wherein the shape sensing unit comprises:
at least one optical fiber comprising a characteristic geometric structure disposed proximate to the first joint configured to bend in response to a third plurality of physical perturbations;
a light transmitting unit coupled to the one optical fiber, wherein the transmitting unit is configured to provide at least one light pulse to the one optical fiber;
a light sensing unit coupled to the one optical fiber, wherein the light sensing unit is configured to receive reflected light from the one optical fiber in response to the characteristic geometric structure bending; and
a shape sensing unit processor coupled to the light sensing unit, wherein the shape sensing unit processor is configured to determine curvature data associated with the one optical fiber in response to the reflected light; and
a central processing device coupled to the plurality of inertial sensing units and to the shape sensing unit, wherein the central processing device is configured to determine movement associated with the plurality of segments in response to the orientation data, the velocity data and the curvature data.

2. The system in accordance with claim 1, wherein the shape sensing unit comprises a housing configured to house the light transmitting unit, the light sensing unit, and the shape sensing unit processor.

3. The system in accordance with claim 1
wherein the plurality of inertial sensing units comprising a third inertial sensing unit;
wherein the plurality of segments comprises a third segment;
wherein the third inertial sensing unit is coupled to the third segment;
wherein the second segment is coupled to the second segment via a second joint; and
wherein the shape sensing unit is also coupled to the the second joint and the third segment.

4. The system in accordance with claim 1
wherein the first inertial sensing unit is coupled to a sacrum of a user;
wherein the second inertial sensing unit is coupled to a sternum of the user; and
wherein the one optical fiber is disposed along a spine of the user.

5. The system in accordance with claim 1 wherein the plurality of inertial sensing units and the shape sensing units are synchronized to a common reference signal.

6. The system in accordance with claim 1 further comprising:
a temperature sensor coupled to a body of a user, wherein the temperature sensor is configured to determine a body temperature; and
wherein the central processing device is coupled to the temperature sensor and is configured to determine the movement associated with the plurality of segments also in response to the body temperature.

7. The system in accordance with claim 1
wherein the shape sensing unit comprises a temperature sensor configured to determine an operating temperature; and
wherein the central processing device is coupled to the temperature sensor and is configured to determine the movement associated with the plurality of segments also in response to the operating temperature.

8. The system in accordance with claim 1 wherein each sensing unit from the plurality of inertial sensing units comprises a wireless data transmitter configured to transmit the orientation data and the velocity data to the central processing device.

9. The system in accordance with claim 1 wherein characteristic geometric structure comprises a grating structure.

10. The system in accordance with claim 9 wherein the grating structure comprises a Fiber Bragg Grating structure.

11. A method for capturing motion comprising:
capturing with a first accelerometer of a first inertial sensing unit coupled to a first portion of a user body, first acceleration data associated with the first portion of the user body in response to a first set of perturbations from a plurality of physical perturbations of the user;
capturing with a first gyroscope of the first inertial sensing unit, first rotation data associated with the first portion of the user body in response to a second set of perturbations from the plurality of physical perturbations of the user;
determining with a first inertial processor of the first inertial sensing unit, first orientation data and first velocity data associated with the first portion of the user body in response to the first acceleration data and to the first rotation data;
capturing with a second accelerometer of a second inertial sensing unit coupled to a second portion of a user body, second acceleration data associated with the second portion of the user body in response to a third set of perturbations from the plurality of physical perturbations of the user, wherein the first portion and the second portion are coupled via a joint portion of the user body;
capturing with a second gyroscope of the second inertial sensing unit, second rotation data associated with the second portion of the user body in response to a fourth set of perturbations from the plurality of physical perturbations of the user;
determining with a second inertial processor of the second inertial sensing unit, second orientation data and second velocity data associated with the second portion of the user body in response to the second acceleration data and to the second rotation data;
outputting with a light transmitting unit of a shape sensing unit , a light signal to at least one optical fiber of the shape sensing unit, wherein at least a portion of the one optical fiber is disposed proximate to the joint portion of the user body;
sensing with a light sensing unit of the shape sensing unit, reflected light signals from the one optical fiber in response to the portion of the one optical fiber bending in response to a fifth set of perturbations from the plurality of physical perturbations of the user;
determining with a shape sensing processor of the shape sensing unit, curvature data associated with the joint portion of the user body in response to the reflected light signals; and
determining with a remote processor movement associated with the user in response to the first orientation data, the first velocity data, the second orientation data, the second velocity data and the curvature data.

12. The method of claim 11 further comprising:
receiving the first inertial sensing unit;
coupling the first inertial sensing unit to the first portion of the user body;
receiving the shape sensing unit; and
disposing the portion of the one optical fiber proximate to the joint portion of the user body.

13. The method of claim 11 further comprising:
capturing with a third accelerometer of a third inertial sensing unit coupled to a third portion of a user body, third acceleration data associated with the third portion of the user body in response to a sixth plurality of perturbations from the plurality of physical perturbations of the user, wherein the second portion and the third portion are coupled via another joint portion of the user body;
capturing with a third gyroscope of the third inertial sensing unit, third rotation data associated with the third portion of the user body in response to a seventh set of perturbations from the plurality of physical perturbations of the user;
determining with a third inertial processor of the third inertial sensing unit, third orientation data and third velocity data associated with the third portion of the user body in response to the third acceleration data and to the third rotation data; and
wherein the determining with the remote processor the movement associated with the user is in response to the first orientation data, the first velocity data, the second orientation data, the second velocity data, the third orientation data, the third velocity data, and the curvature data.

14. The method of claim 11 further comprising:
disposing the first inertial sensing unit proximate to a sacrum of the user;
disposing the second inertial sensing unit proximate to a sternum of the user; and
disposing the one optical fiber along a spine of the user.

15. The method of claim 11 further comprising synchronizing the plurality of inertial sensing units and the shape sensing units are synchronized to a common reference signal.

16. The method of claim 11 further comprising:
measuring with a temperature sensor coupled to the body of a user, a body temperature; and
wherein the determining with the shape sensing processor , the curvature data associated with the joint portion of the user body is also in response to the operating temperature.

17. The method of claim 11 further comprising:
measuring with a temperature sensor coupled to the one optical fiber, an operating temperature; and
wherein the determining with the shape sensing processor , the curvature data associated with the joint portion of the user body is also in response the operating temperature.

18. The method of claim 11 further comprising transmitting with a wireless transmitter of the first inertial sensing unit, the first orientation data and the first velocity data to the fourth processor.

19. The method of claim 11 wherein at least a portion of the one optical fiber is comprises a grating structure.

20. The method of claim 11 wherein the grating structure comprises a Fiber Bragg Grating structure.
